(19) United States
(12) Patent Application Publication
(10) Pub. No.: US 2024/0148616 A1
(43) Pub. Date: May 9, 2024

(54) WATER-IN-OIL EMULSION COMPOSITION, COSMETIC, AND METHOD FOR PREPARING WATER-IN-OIL EMULSION COMPOSITION

(71) Applicant: SHISEIDO COMPANY, LTD., Tokyo (JP)

(72) Inventors: Takehito SHISHIDO, Tokyo (JP); Kei UJIMOTO, Tokyo (JP); Ryushi FUKUHARA, Tokyo (JP)

(21) Appl. No.: 18/548,273

(22) PCT Filed: Mar. 17, 2022
(86) PCT No.: PCT/JP2022/012473

§ 371 Date: Aug. 29, 2023

(30) Foreign Application Priority Data
Mar. 29, 2021 (JP) 2021-055610

(51) Int. Cl. A61K 8/06; A61K 8/41; A61K 8/58; A61Q 19/00

(52) U.S. Cl. CPC A61K 8/064; A61K 8/416; A61K 8/585; A61Q 19/00; A61K 2800/10; A61K 2800/612; A61K 2800/80

(57) ABSTRACT

A water-in-oil emulsion composition includes:
an external oil phase, in which a first hydrophobic powder is dispersed; and
an internal aqueous phase, in which a second hydrophobic powder is dispersed,
in which, among the first hydrophobic powder and the second hydrophobic powder, at least the second hydrophobic powder is a metal oxide treated with trimethylethoxysilane,
in which the water-in-oil emulsion composition further includes a dispersant that disperses the second hydrophobic powder in the internal aqueous phase, and
in which the dispersant is at least one of a silicone-based surfactant and a cationic surfactant.

### TECHNICAL FIELD

The present invention relates to a water-in-oil emulsion composition, a cosmetic, and a method for preparing the water-in-oil emulsion composition.

### BACKGROUND ART

A cosmetic such as a liquid foundation contains an oil phase such as an oil agent, and an aqueous phase such as an aqueous component, and metal oxide powders such as a UV scattering agent, a pigment, and so forth are dispersed in the oil phase in order to impart various functions to the cosmetic, including UV protection capacity, color development, and covering power. In addition, these metal oxide powders are subject to hydrophobization treatment, which blocks the surface catalytic activity of these metal oxides, so that water resistance and long-lasting effects are imparted to the cosmetic (see, for example, patent documents 1 and 2).

### CITATION LIST

### Patent Documents

Patent Document 1: Unexamined Japanese Patent Application Publication No. 2019-073460

Patent Document 2: Unexamined Japanese Patent Application Publication No. 2015-168635

### SUMMARY OF INVENTION

### Technical Problem

Since the surface of metal oxide powders is hydrophobized, they disperse in the oil phase but do not disperse well in the aqueous phase. That is, it is difficult to disperse metal oxide powders throughout cosmetics. Therefore, conventional cosmetics are limited in various functions such as UV protection capacity.

It is therefore an object of the present invention to provide a water-in-oil emulsion composition that can improve various functions of cosmetics.

### Solution to Problem

The water-in-oil emulsion composition according to one aspect of the present invention includes:
an external oil phase, in which a first hydrophobic powder is dispersed; and
an internal aqueous phase, in which a second hydrophobic powder is dispersed,
in which, among the first hydrophobic powder and the second hydrophobic powder, at least the second hydrophobic powder is a metal oxide treated with trimethylethoxysilane,
in which the water-in-oil emulsion composition further includes a dispersant that disperses the second hydrophobic powder in the internal aqueous phase, and
in which the dispersant is at least one of a silicone-based surfactant and a cationic surfactant.

### Advantageous Effects of the Invention

According to one aspect of the present invention, various functions of cosmetics can be improved.

### BRIEF DESCRIPTION OF DRAWINGS

FIG. 1 is a schematic diagram that illustrates a water-in-oil emulsion composition;

FIG. 2 is a schematic diagram that illustrates how a water-in-oil emulsion composition is used;

FIG. 3 is a flowchart that shows an example method for preparing a water-in-oil emulsion composition;

FIG. 4 is a flowchart that shows an example of a second dispersing step in a method for preparing a water-in-oil emulsion composition;

FIG. 5 is a graph that shows the absorbance of a water-in-oil emulsion composition;

FIG. 6 is a graph that shows the absorbance of a water-in-oil emulsion composition; and

FIG. 7 is a graph that shows the absorbance of a water-in-oil emulsion composition.

### DESCRIPTION OF EMBODIMENTS

Hereinafter, embodiments of the present invention will be described in detail.

### <Water-in-Oil Emulsion Composition>

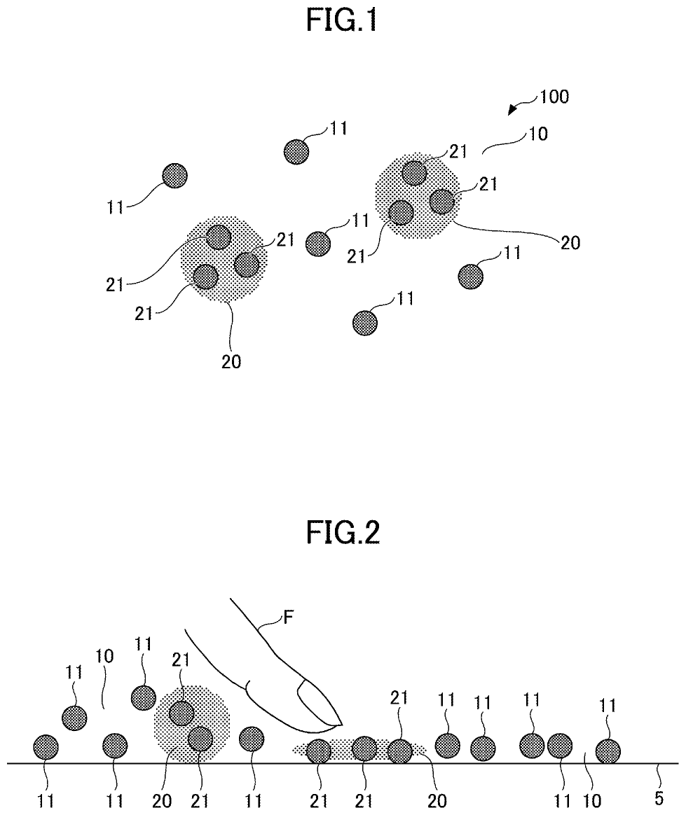

FIG. 1 is a schematic diagram that illustrates a water-in-oil emulsion composition. The water-in-oil emulsion composition according to this embodiment has: an external oil phase, in which a first hydrophobic powder is dispersed; and an internal aqueous phase, in which a second hydrophobic powder is dispersed. To be more specific, as illustrated in FIG. 1, a water-in-oil emulsion composition 100 includes an external oil phase 10 and an internal aqueous phase 20. A first hydrophobic powder 11 is dispersed in the external oil phase 10, and a second hydrophobic powder 21 is dispersed in the internal aqueous phase 20.

As used herein, a water-in-oil emulsion composition refers to an emulsion, in which the oil phase is the continuous phase (external phase) and the aqueous phase is the dispersed phase (internal phase). In addition, the external oil phase is the continuous phase (external phase), which is the oil phase, and the internal aqueous phase is the dispersed phase (internal phase), which is the aqueous phase.

In the water-in-oil emulsion composition of the present embodiment, the composition of the external oil phase is not fixed, and may be composed of, for example, oil components (oils). Examples of the oil components (oils) include volatile oils, non-volatile oils, and so forth.

A volatile oil refers to an oil component that evaporates at room temperature. Examples of volatile oils include hydrocarbon oils such as isododecane, chain silicone oils such as dimethylpolysiloxane (dimethicone), methylphenylpolysiloxane, methylhydrogenpolysiloxane, and so forth. Only one of these volatile oils may be used, or two or more of these volatile oils may be mixed and used.

A non-volatile oil refers to an oil component that does not evaporate at room temperature. Examples of non-volatile oils include paraffin, liquid paraffin, ozokerite, squalane, squalene, pristane, ceresin, vaseline, glyceryl tri(2-ethylhexanoate), isopropyl myristate, cetyl octanoate, octyldodecyl myristate, isopropyl palmitate, octyl palmitate, octyl isopalmitate, butyl stearate, hexyl laurate, myristyl myristate, decyl oleate, hexyldecyl dimethyloctanoate, cetyl lactate, myristyl lactate, lanolin acetate, isocetyl stearate, isopropyl isostearate, isocetyl isostearate, cholesteryl 12-hydroxystearate, ethylene glycol di(2-ethylhexanoate), dipentaerythritol fatty acid ester, N-alkyl glycol monoisostearate, neopentyl glycol dicaprate, diisostearyl malate, glyceryl di(2-heptylundecanoate), trimethylolpropane tri(2-ethylhexylate), trimethylolpropane triisostearate, pentaneerythritol tetra(2-ethylhexylate), glyceryl tri(2-ethylhexylate), trimethylolpropane triisostearate, cetyl-2-ethylhexanoate, 2-ethylhexyl palmitate, glyceryl trimyristate, tri-2-heptylundecanoic acid glyceride, castor oil fatty acid methyl ester, oleic oil, acetoglyceride, 2-heptylundecyl palmitate, diisobutyl adipate, N-lauroyl-L-glutamic acid-2-ctyldodecyl ester, di-2-heptylundecyl adipate, ethyl laurate, diethyl sebacate, diisopropyl sebacate, di-2-ethylhexyl sebacate, 2-hexyldecyl myristate, 2-hexyldecyl palmitate, 2-hexyldecyl adipate, 2-ethylhexyl succinate, ethyl acetate, butyl acetate, amyl acetate, triethyl citrate, crotamiton (C₁₃H₁₇NO), and so forth. Only one of these non-volatile oils may be used, or two or more of these non-volatile oils may be mixed and used.

The content of oil components in the external oil phase is not particularly limited, but is 10% or more by weight and 80% or less by weight of the water-in-oil emulsion composition, preferably 20% or more by weight and 70% or less by weight of the water-in-oil emulsion composition, more preferably 30% or more by weight and 60% or less by weight of the water-in-oil emulsion composition. If the content of oil components in the external oil phase is in this range, the usability of cosmetics using the water-in-oil emulsion composition is less likely to deteriorate.

The external oil phase of the water-in-oil emulsion composition may contain components other than those mentioned above, such as a colorant, a stabilizer, a surfactant excluding the dispersant described later, an antioxidant, and a preservative, as long as the effects of the present invention are not impaired.

In the water-in-oil emulsion composition of the present embodiment, the composition of the internal aqueous phase is not fixed, and may be composed of aqueous components, for example. An aqueous component refers to a component that is readily soluble in water. Examples of aqueous components include water, a lower alcohol such as ethanol, polyhydric alcohol such as glycerin, and so forth. Only one of these aqueous components may be used, or two or more of these aqueous components may be mixed and used.

The content of aqueous components is not particularly limited, but is 1% or more by weight and 50% or less by weight of the water-in-oil emulsion composition, preferably 10% or more by weight and 40% or less by weight of the water-in-oil emulsion composition, more preferably 20% or more by weight and 30% or less by weight of the water-in-oil emulsion composition. If the content of aqueous components is in this range, the usability of cosmetics using the water-in-oil emulsion composition is less likely to deteriorate.

The internal aqueous phase of the water-in-oil emulsion composition may contain components other than those mentioned above, such as a colorant, a stabilizer, a surfactant excluding the dispersant described later, an antioxidant, and a preservative, as long as the effects of the present invention are not impaired.

Of the first hydrophobic powder and the second hydrophobic powder, at least the second hydrophobic powder is a metal oxide that is treated with trimethylethoxysilane. Note that examples of hydrophobic powders include a powder obtained by hydrophobizing the surface of a hydrophilic powder, a powder obtained by further hydrophobizing the surface of a hydrophobic powder, and so forth. Here, a hydrophobic powder refers to a powder that is dispersed in the hexane phase when the powder is dispersed in a mixed solvent of water and hexane at a mass ratio of 1:1.

In the water-in-oil emulsion composition of the present embodiment, both the first hydrophobic powder and the second hydrophobic powder are composed of metal oxides treated with trimethylethoxysilane. Note that, in the water-in-oil emulsion composition of the present embodiment, only the second hydrophobic powder may be composed of a metal oxide treated with trimethylethoxysilane.

The metal oxides are not particularly limited, and examples thereof include inorganic metal oxides, organic metal oxides, composite metal oxides, and so forth. Since the surface of metal oxides is hydrophobized, they disperse well in the oil phase as is, but do not disperse well in the aqueous phase.

Examples of inorganic metal oxides include titanium oxide, black titanium oxide, conjo, ultramarine, bengara, yellow iron oxide, black iron oxide, zinc oxide, aluminum oxide, magnesium oxide, zirconium oxide, magnesium carbonate, calcium carbonate, carbon black, aluminum silicate, magnesium silicate, aluminum magnesium silicate, mica, synthetic mica, synthetic sericite, sericite, talc, silicon carbide, barium sulfate, boron nitride, and so forth.

Examples of organic metal oxides include nylon powder, acrylic powder, silk powder, crystalline cellulose, N-acyl lysine, tar pigments, and so forth.

Examples of composite metal oxides include bismuth oxychloride, mica titanium, iron oxide-coated mica, iron oxide mica titanium, mica titanium treated with organic pigment, aluminum powder, and so forth.

One or more of these metal oxides can be used. As metal oxides to use as hydrophobic powders, titanium oxide, zinc oxide, and aluminum oxide, whose surface is hydrophobized with trimethylethoxysilane, are preferable. Among these, zinc oxide is particularly preferable.

The treatment agent used for the hydrophobization treatment of metal oxides (hereinafter referred to as “hydrophobization treatment agent”) is trimethylethoxysilane. For example, alkylalkoxysilanes such as octyltriethoxysilane, octyltrimethoxysilane, and so forth can be used. One or more of these treatment agents can be used. These treatment agents may also be used in combination with aluminum hydroxide.

The method of treating powder with these hydrophobizing agents is not particularly limited, but, for example, a wet method using a solvent, a vapor phase method, a mechanochemical method, and so forth can be used. In addition, the amount of the hydrophobizing agent upon treatment is not particularly limited, but, considering the adhesion to the skin, the uniformity of the finish, and the longevity of the makeup when the water-in-oil emulsion composition is used for cosmetics, it is preferably 0.5% or more by weight and 7% or less by weight of the hydrophobic powder, more preferably 1% or more by weight and 5% or less by weight by weight of the hydrophobic powder.

The content of the hydrophobic powder is not particularly limited, but the first hydrophobic powder and the second hydrophobic powder are both preferably 0.1% or more by weight and 35% or less by weight, more preferably 0.5% or more by weight and 30% or less by weight, more preferably 1% or more by weight and 25% or less by weight. If the content of the hydrophobic powder is in this range, when the water-in-oil emulsion composition is used in cosmetics, the makeup is less likely to become uneven, and the layer of the makeup is more likely to become even and less likely to fall in the pores.

The water-in-oil emulsion composition of the present embodiment contains a dispersant that disperses the second hydrophobic powder composed of the above-mentioned metal oxide in the internal aqueous phase. The dispersant is at least one of a silicone-based surfactant and a cationic surfactant.

Note that the dispersant may disperse the first hydrophobic powder in the external oil phase. The dispersant used in this embodiment disperses the first hydrophobic powder in the external oil phase and disperses the second hydrophobic powder in the internal aqueous phase.

A silicone-based surfactant is a surfactant that has an organosiloxane structure in its molecular structure. Examples of the silicone-based surfactant include a polyglyceryl-modified silicone, glyceryl modified silicone, sugar-modified silicone, fluorine polyether-modified silicone, polyether-modified silicone, carboxylic acid-modified silicone, a mixture of a carboxylic acid-modified silicone and an alkanolamine, sugar-modified silicone, linear silicone/polyether block copolymer (polysilicone-13, etc.), long-chain alkyl-polyether co-modified silicone, and so forth. Among these, the mixture of a carboxylic acid-modified silicone and an alkanolamine is preferable. Only one of these silicone-based surfactants may be used, or two or more of these silicone-based surfactants may be mixed and used.

A cationic surfactant refers to a surfactant having a cationic hydrophilic group. The cationic surfactant is not particularly limited, but it is preferable to use one or more first surfactants selected from quaternary ammonium salts, more preferably a quaternary ammonium salt cationic surfactant, still more preferably a dialkyldimethylammonium chloride represented by the following general formula:

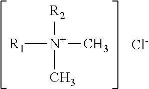

(in the above formula, R1 is an alkyl group or a benzyl group having 10 to 22 carbon atoms, R2 is a methyl group or an alkyl group having 10 to 22 carbon atoms, R3 and R4 are an alkyl group or a hydroxyalkyl group having 1 to 3 carbon atoms, and X is a halogen atom or a methylsulfate residue).

Examples of dialkyldimethylammonium chloride represented by the above general formula include distearyldimethylammonium chloride, didecyldimethylammonium chloride, and so forth. Among these, distearyldimethylammonium chloride is preferable.

The content of the dispersant is not particularly limited, but is preferably 0.1% or more by weight and 5% or less by weight of each of the first hydrophobic powder and the second hydrophobic powder, more preferably 0.1% or more by weight and 4% or less by weight of each of the powders, still more preferably 0.1% or more by weight and 3% or less by weight of each of the powders. If the content of the dispersant is in this range, both the dispersibility of the first hydrophobic powder in the external oil phase and the dispersibility of the second hydrophobic powder in the internal aqueous phase can be improved.

Furthermore, in the water-in-oil emulsion composition of the present embodiment, the internal aqueous phase contains an aqueous slurry, and this aqueous slurry contains the second hydrophobic powder, an aqueous medium, and the above-mentioned dispersant that disperses the second hydrophobic powder in the aqueous medium. As used herein, an aqueous slurry refers to a suspension in which powder is dispersed in an aqueous medium.

The aqueous medium is a medium containing water, and may contain other components than water. Components other than water may include alcohol and the like.

Although the content of the aqueous medium is not particularly limited, it is preferably 50% or more by weight and 90% or less by weight of the aqueous slurry, more preferably 55% or more by weight and 85% or less by weight of the aqueous slurry, still more preferably 60% or more by weight and 80% or less by weight of the aqueous slurry. If the content of the aqueous medium is in this range, the suspension of the aqueous slurry can be stabilized.

Although the content of the dispersant is not particularly limited, it is preferably 0.1% or more by weight and 5% or less by weight of the aqueous slurry, more preferably 0.1% or more by weight and 4% or less by weight of the aqueous slurry, still more preferably 0.1% or more by weight 3% or less by weight of the aqueous slurry. If the content of the dispersant is in this range, it is possible to improve the dispersibility of the second hydrophobic powder in the internal aqueous phase when the aqueous slurry is dispersed in the internal aqueous phase.

The form of the aqueous slurry contained in the internal aqueous phase is not particularly limited, but it is preferable to use an aqueous slurry in which the second hydrophobic powder is dispersed in a mixed solvent of a dispersant and an aqueous medium. Here, the mixed solvent is a solvent obtained by adding and mixing a dispersant in an aqueous medium.

Furthermore, as for the nature of the aqueous slurry contained in the internal aqueous phase, an aqueous slurry in which a mixture of the second hydrophobic powder and a dispersant is dispersed in an aqueous medium is more preferable. Here, the mixture is the second hydrophobic powder impregnated with a dispersant.

Note that the mixture dispersed in the aqueous slurry may contain an alcohol as another component. The alcohol is not particularly limited, and may be a lower alcohol, polyhydric alcohol, or the like.

Examples of the lower alcohol include ethanol, propanol, isopropanol, isobutyl alcohol, t-butyl alcohol, and so forth.

Examples of the polyhydric alcohol include: dihydric alcohol (for example, ethylene glycol, propylene glycol, trimethylene glycol, 1,2-butylene glycol, 1,3-butylene glycol, tetramethylene glycol, 2,3-butylene glycol, pentamethylene glycol, 2-butene-1,4-diol, hexylene glycol, octylene glycol, etc.); trihydric alcohol (for example, glycerin, trimethylolpropane, etc.); tetrahydric alcohol (for example, pentaerythritol such as 1,2,6-hexanetriol, etc.); pentahydric alcohol (for example, xylitol, etc.); hexahydric alcohol (for example, sorbitol, mannitol, etc.); polyhydric alcohol polymer (for example, diethylene glycol, dipropylene glycol, triethylene glycol, polypropylene glycol, tetraethylene glycol, diglycerin, polyethylene glycol, triglycerin, tetraglycerin, polyglycerin, etc.); dihydric alcohol alkyl ethers (for example, ethylene glycol monomethyl ether, ethylene glycol monoethyl ether, ethylene glycol monobutyl ether, ethylene glycol monophenyl ether, ethylene glycol monohexyl ether, ethylene glycol mono-2-methylhexyl ether, ethylene glycol isoamyl ether, ethylene glycol benzyl ether, ethylene glycol isopropyl ether, ethylene glycol dimethyl ether, ethylene glycol diethyl ether, ethylene glycol dibutyl ether, etc.); dihydric alcohol alkyl ethers (for example, diethylene glycol monomethyl ether, diethylene glycol monoethyl ether, diethylene glycol monobutyl ether, diethylene glycol dimethyl ether, diethylene glycol diethyl ether, diethylene glycol butyl ether, diethylene glycol methyl ethyl ether, triethylene glycol monomethyl ether, triethylene glycol monoethyl ether, propylene glycol monomethyl ether, propylene glycol monoethyl ether, propylene glycol monobutyl ether, propylene glycol isopropyl ether, dipropylene glycol methyl ether, dipropylene glycol ethyl ether, dipropylene glycol butyl ether, etc.); dihydric alcohol ether ester (for example, ethylene glycol monomethyl ether acetate, ethylene glycol monoethyl ether acetate, ethylene glycol monobutyl ether acetate, ethylene glycol monophenyl ether acetate, ethylene glycol diazibate, ethylene glycol disuccinate, diethylene glycol monoethyl ether acetate, diethylene glycol monobutyl ether acetate, propylene glycol monomethyl ether acetate, propylene glycol monoethyl ether acetate, propylene glycol monopropyl ether acetate, propylene glycol monophenyl ether acetate, etc.); glycerin monoalkyl ether (for example, xyl alcohol, selachyl alcohol, batyl alcohol, etc.); sugar alcohol (for example, sorbitol, maltitol, maltotriose, mannitol, sucrose, erythritol, glucose, fructose, amylolytic sugar, maltose, xylitose, starch-degrading sugar reducing alcohol, etc.); glysolid; tetrahydrofurfuryl alcohol; POE-tetrahydrofurfuryl alcohol; POP-butyl ether; POP/POE-butyl ether; tripolyoxypropylene glycerin ether; POP-glycerin ether; POP-glycerol ether phosphate; POP/POE-pentaneerythritol ether; and polyglycerin.

The content of the alcohol is not particularly limited, but is preferably 1% or more by weight and 25% or less by weight of the aqueous slurry, more preferably 3% or more by weight and 20% or less by weight of the aqueous slurry, still more preferably 5% or more by weight and 15% or less by weight of the aqueous slurry. If the content of the alcohol is in this range, the dispersibility of the hydrophobic powder in the aqueous slurry can be stabilized.

The water-in-oil emulsion composition of the present embodiment may include, in addition to the above components, an emulsifier (excluding the above-described surfactant), a thickener (including a stabilizer), a moisturizer (including a preservative), a colorant, and an antioxidant, as long as the effects of the present invention are not impaired.

Examples of the emulsifier include hydrogenated lecithin, sorbitan laurate, sorbitan palmitate, sorbitan oleate, sorbitan stearate, sorbitan isostearate, sorbitan sesquioleate, sorbitan trioleate, sorbitan tristearate, sorbitan sesquiisostearate, and so forth.

The content of the emulsifier is not particularly limited, but is 0.01% or more by weight and 20% or less by weight of the water-in-oil emulsion composition, preferably 0.01% or more by weight and 10% or less by weight of the water-in-oil emulsion composition, and more preferably is 0.01% or more by weight and 5% or less by weight of the water-in-oil emulsion composition.

Examples of the thickener include a dextrin fatty acid ester, sucrose fatty acid ester, cellulose derivatives, aluminum isostearate, dibutylethylhexanoyl glutamide, dibutyl lauroyl glutamide, bisdialkyl (C14-18) amide (ethylenediamine/hydrogenated dimer dilinoleic acid) copolymers, (VP/eicosene) copolymer, and so forth. Only one of these stabilizers may be used, or two or more of these stabilizers may be mixed and used.

The content of the thickener is not particularly limited, but is 0.001% or more by weight and 3% or less by weight of the water-in-oil emulsion composition, preferably 0.001% or more by weight and 1.5% or less by weight of the water-in-oil emulsion composition, and more preferably 0.001% or more by weight and 1% or less by weight of the water-in-oil emulsion composition.

Examples of the moisturizer include polyhydric alcohols (1,3-butylene glycol, etc.), paraoxybenzoic acid ester, phenoxyethanol, octoxyglycerin, benzoic acid, salicylic acid, carbolic acid, sorbic acid, parachlormetacresol, hexachlorophene, benzalkonium chloride, chlorhexidine chloride, trichlorocarbanilide, and so forth.

The content of the moisturizer is not particularly limited, but is 0.02% or more by weight and 40% or less by weight of the water-in-oil emulsion composition, preferably 0.2% or more by weight and 30% or less by weight of the water-in-oil emulsion composition, more preferably 1% or more by weight and 20% or less by weight of the water-in-oil emulsion composition.

A colorant is a component that colors the aqueous slurry (that is, colors the cosmetics). Examples of the colorant include a red iron oxide, yellow iron oxide, black iron oxide, inorganic white pigments such as zinc oxide, inorganic red pigments such as iron titanate, mango violet, inorganic purple pigments such as cobalt violet, chromium oxide, chromium hydroxide, inorganic green pigments such as cobalt titanate, ultramarine, inorganic blue pigments such as Prussian blue, aluminum powder, metal powder pigments such as copper powder, zirconium, organic pigments such as barium or aluminum lakes, chlorophyll, natural pigments such as β-carotene, and so forth. Only one of these colorants may be used, or two or more of these colorants may be mixed and used.

The content of the colorant is not particularly limited, but is 0.1% or more by weight and 40% or less by weight of the aqueous slurry, preferably 0.3% or more by weight and 35% or less by weight of the aqueous slurry, more preferably 0.5% by weight and 30% or less by weight of the aqueous slurry.

Examples of the antioxidant include ascorbic acid, α-tocopherol, dibutyl hydroxytoluene, butyl hydroxyanisole, and so forth.

As described above, in the water-in-oil emulsion composition of the present embodiment, at least the second hydrophobic powder out of the first hydrophobic powder and the second hydrophobic powder is a metal oxide treated with trimethylethoxysilane, and at least one of a silicone-based surfactant and a cationic surfactant is included as a dispersant for dispersing the second hydrophobic powder in the internal aqueous phase. As a result of this, the hydrophobic powder can be dispersed throughout the water-in-oil emulsion composition (see FIG. 1). Moreover, in cosmetics using such a water-in-oil emulsion composition, various functions such as ultraviolet protection capacity, color development, covering power, and so forth can be improved.

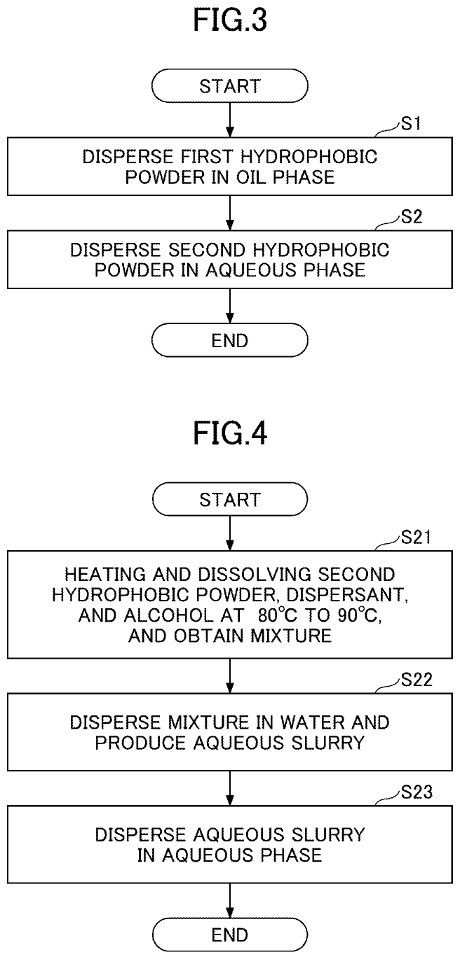

For example, as illustrated in FIG. 3, when a cosmetic containing a water-in-oil emulsion composition is applied to the skin S with a finger F, the first hydrophobic powder 11 and the second hydrophobic powder 21 can cover the entire skin S even if the external oil phase 10 or the internal aqueous phase 20 is exposed on the surface of the skin S.

As described above, in the water-in-oil emulsion composition of the present embodiment, an aqueous slurry containing a second hydrophobic powder, an aqueous medium, and the above-described dispersant that disperses the second hydrophobic powder in the aqueous medium is contained in the internal aqueous phase, so that it becomes easy to disperse the second hydrophobic powder in the internal aqueous phase.

In the water-in-oil emulsion composition of the present embodiment, as described above, an aqueous slurry, in which the second hydrophobic powder is dispersed in the mixed solvent of the dispersant and the aqueous medium, is contained in the internal aqueous phase, so that it becomes easier to disperse the second hydrophobic powder in the internal aqueous phase.

In the water-in-oil emulsion composition of the present embodiment, as described above, an aqueous slurry, in which a mixture of the second hydrophobic powder and the dispersant is dispersed in an aqueous medium, is contained in the internal aqueous phase, so that it becomes easier to disperse the second hydrophobic powder in the internal aqueous phase.

In the water-in-oil emulsion composition of the present embodiment, as described above, when a silicone-based surfactant is used as a dispersant, among the silicone-based surfactants, a mixture of carboxylic acid-modified silicone and alkanolamine may be used, so that it becomes possible to further improve the dispersibility of the hydrophobic powder in the aqueous phase when an aqueous slurry is dispersed in the aqueous phase.

With the water-in-oil emulsion composition of the present embodiment, as described above, when a cationic surfactant is used as a dispersant, among the cationic surfactants, dialkyldimethylammonium chloride represented by the above general formula may be used, so that it becomes possible to further improve the dispersibility of the hydrophobic powder in the aqueous phase when an aqueous slurry is dispersed in the aqueous phase.

As described above, the water-in-oil emulsion composition of the present embodiment can improve the emulsification of the external oil phase and the internal aqueous phase in the water-in-oil emulsion composition by containing an emulsifier.

As described above, the water-in-oil emulsion composition of the present embodiment can stabilize the emulsification of the external oil phase and the internal aqueous phase in the water-in-oil emulsion composition by containing a thickener.

As described above, the water-in-oil emulsion composition of the present embodiment can achieve a preservation effect for the water-in-oil emulsion composition by containing a moisturizer.

### <Method for Preparing Water-in-Oil Emulsion Composition>

FIG. 3 is a flowchart that shows an example method for preparing a water-in-oil emulsion composition according to this embodiment. The method for preparing a water-in-oil emulsion composition according to the present embodiment is a method for preparing a water-in-oil emulsion composition in which the aqueous phase is dispersed in the oil phase, and includes a first dispersing step of dispersing a first hydrophobic powder in the oil phase, and a second dispersing step of dispersing a second hydrophobic powder in the aqueous phase (see FIG. 3, and steps S1 and S2).

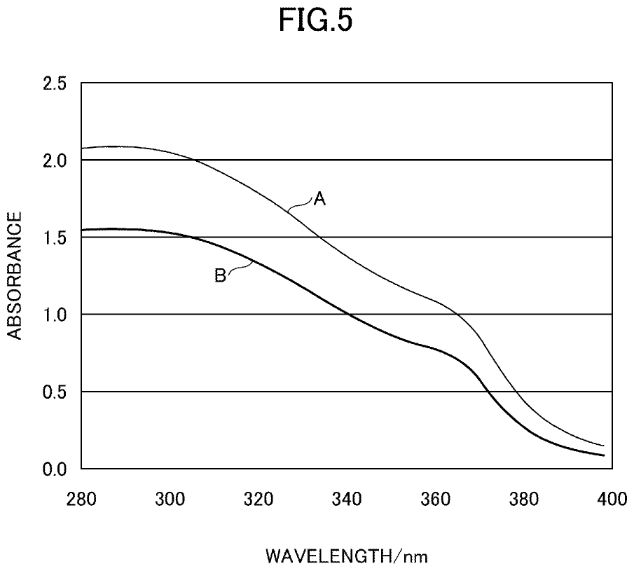

In the method for preparing a water-in-oil emulsion composition according to the present embodiment, as illustrated in FIG. 4, the second dispersing step further includes heating and dissolving the second hydrophobic powder, a dispersant, and an alcohol at 80° C. to 90° C. to obtain a mixture, dispersing the mixture in water to produce an aqueous slurry, and dispersing the aqueous slurry in the aqueous phase.

To be more specific, a dispersant and an alcohol are placed in a container such as a disper mixer, and heated and dissolved at 85° C., and the resulting solution is stirred while adding the second hydrophobic powder to prepare a mixture (see FIG. 4 and step S21). Furthermore, water is added to this mixture, followed by stirring and dispersion with a homogenizer at 7000 rpm, for 2 minutes, to obtain an aqueous slurry (see FIG. 2 and step S22). Note that the stirring time is not fixed, and may be, for example, approximately 1 minute to 30 minutes.

Then, the resulting aqueous slurry is added to an aqueous phase and dispersed by stirring it with a homogenizer at 7000 rpm, for 2 minutes, thereby obtaining an aqueous phase in which the second hydrophobic powder is dispersed (see FIG. 4 and step S23). Note that the stirring time is not fixed, and may be, for example, approximately 1 minute to 30 minutes. By adding this aqueous phase in which the second hydrophobic powder is dispersed, to the oil phase in which the first hydrophobic powder is dispersed, and emulsifying the mixture by stirring it with a homogenizer at 7000 rpm, for 2 minutes, a water-in-oil emulsion composition is obtained in which the oil phase is the continuous phase (external phase) and the aqueous phase is the dispersed phase (internal phase).

In the method for preparing a water-in-oil emulsion composition according to the present embodiment, as described above, the first hydrophobic powder is dispersed in the external oil phase, and the second hydrophobic powder is also dispersed in the internal aqueous phase, so that a water-in-oil emulsion composition in which hydrophobic powders are dispersed throughout the water-in-oil emulsion composition can be obtained. By using such a water-in-oil emulsion composition in cosmetics, it is possible to improve various functions of cosmetics such as ultraviolet protection capacity, color development, and covering power.

### <Cosmetic>

The cosmetic according to this embodiment contains the water-in-oil emulsion composition described above. That is, the cosmetic of the present embodiment contains the above-described aqueous slurry, and, to be more specific, contains the water-in-oil (W/O) emulsion composition shown in FIG. 1.

The form of the cosmetic according to the present embodiment is not particularly limited, and may be liquid, milky lotion, paste, cream, gel, and so forth.

The use of the cosmetic according to this embodiment is not particularly limited, and can be used in, for example: makeup cosmetics such as makeup base, liquid foundation, cream foundation, concealer, blusher, eye shadow, mascara, eyeliner, eyebrow, overcoat agent, and lipstick; UV protection cosmetics such as sunscreen lotion, and sunscreen cream; and skin care cosmetics such as skin care milky lotion, skin care cream, BB cream, and beauty essence. Among these, make-up cosmetics and UV protective cosmetics are preferable, and liquid foundations and sunscreen emulsions are more preferable.

The method for manufacturing a cosmetic according to this embodiment is not particularly limited. For example, a water-in-oil emulsion composition (including an aqueous slurry) prepared as described above, with optional additions of other components (colorant, stabilizer, activator, antioxidant, preservative, etc.), is placed in any cosmetic container from which an appropriate amount of cosmetic can be taken out.

As described above, the cosmetic according to the present embodiment contains a water-in-oil emulsion composition having an external oil phase in which a first hydrophobic powder is dispersed, and an internal aqueous phase in which a second hydrophobic powder is dispersed, so that the first hydrophobic powder is dispersed in the external oil phase, and the second hydrophobic powder is also dispersed in the internal aqueous phase. As a result of this, a water-in-oil emulsion composition in which hydrophobic powders are dispersed throughout the water-in-oil emulsion composition can be contained in the cosmetic. Therefore, the cosmetic of the present embodiment can improve various functions of cosmetics such as UV protection capacity, color development, and covering power.

### EMBODIMENTS

The present invention will be further described below using embodiments. Note that, hereinafter, “parts” and “%” are based on mass unless otherwise specified. In addition, various tests and evaluations are conducted according to the following methods.

### <Sample (Water-in-Oil Emulsion Composition)>

A water-in-oil emulsion composition sample was obtained by mixing water, a thickener, a dispersant, an oil component, an ultraviolet protection agent, and a functional powder. To be more specific, a powder was dispersed in the oil component mixed by using a homomixer, and then a well-mixed aqueous component was added to obtain the composition.

Note that the names (manufacturers and product names) of components contained in the water-in-oil emulsion composition are as follows (Tables 1 and 2):
Water (ion-exchanged water);
Disteardimonium hectorite (Benton 38VCG manufactured by Elementis Specialties Inc.);
Polyethylene glycol (PEG)-9 polydimethylsiloxyethyl dimethicone/tocopherol (KF-6038 manufactured by Shin-Etsu Chemical Co., Ltd.);
Distearyldimonium chloride (Cation DSV manufactured by Sanyo Chemical Industries, Ltd.);
Isostearic acid (isostearic acid EX manufactured by KOKYU ALCOHOL KOGYO CO., LTD.);
Polyethylene glycol (PEG)-10 dimethicone (KF-6051 manufactured by Shin-Etsu Chemical Co., Ltd.);
Bisbutyl dimethicone polyglyceryl-3 (KF-6109 manufactured by Shin-Etsu Chemical Co., Ltd.);
Diisopropyl sebacate (Eceran 200 manufactured by NIPPON FINE CHEMICAL CO., LTD);
Dimethicone* (KF-96L-1.5CS manufactured by Shin-Etsu Chemical Co., Ltd.);
Dimethicone** (KF-96A-6T manufactured by Shin-Etsu Chemical Co., Ltd.);
Stearic acid (PALMAC 70-18 manufactured by ACIDCHEM INTERNATIONAL SDN. BHD.);
Isopropyl myristate (IPM-R manufactured by KOKYU ALCOHOL KOGYO CO., LTD.);
Isododecane (ISODODECANE manufactured by INEOS Manufacturing Deutschland GmbH);
Polypropylene glycol (PPG)-17/dibutylhydroxytoluene (BHT) (New Pole (registered trademark) PP-1000 manufactured by Sanyo Chemical Industries, Ltd.);
Trimethylsiloxysilicate/dimethicone (SSD-R-3 manufactured by Shin-Etsu Chemical Co., Ltd.);
Dextrin palmitate (Rheopearl (registered trademark) KL2 manufactured by Chiba Flour Milling Co., Ltd.);
Zinc oxide/triethoxycaprylylsilane (MZX-5080TS manufactured by TAYCA CORPORATION);
Octocrylene (Parsol (registered trademark) 340 manufactured by DSM Nutritional Products Ltd.);
Bisethylhexyloxyphenol methoxyphenyltriazine (Tinosorb (registered trademark) S manufactured by BASF);
Diethylaminohydroxybenzoyl hexyl benzoate (Uvinul A Plus Granular, manufactured by BASF);
Ethylhexyl methoxycinnamate/BHT (Uvinul MC 80 manufactured by BASF);
(Vinyl dimethicone/methicone silsesquioxane) crosspolymer (KSP-100 manufactured by Shin-Etsu Chemical Co., Ltd.);
Talc/dimethicone (SA-Talc JA-68R manufactured by Miyoshi Kasei, Inc.);
Methyl methacrylate crosspolymer (Ganz Pearl (registered trademark) GMX-0810 manufactured by Ganz Chemical Co., Ltd.);
Silica (SUNSPHERE (registered trademark) L-51S manufactured by AGC Si-Tech Co., Ltd.);
Glycerin (concentrated glycerin for cosmetics, manufactured by Sakamoto Yakuhin Kogyo Co., Ltd.);
BG (1,3-butylene glycol manufactured by Daicel Corporation);
Cyclopentasiloxane (Execol D-5 manufactured by Shin-Etsu Chemical Co., Ltd.);
Dimethicone*** (KF-96A-20CS manufactured by Shin-Etsu Chemical Co., Ltd.);
Diphenylsiloxyphenyl trimethicone (KF-56A manufactured by Shin-Etsu Chemical Co., Ltd.);
Titanium oxide/aluminum hydroxide/hydrogen dimethicone (STR-100C-LP manufactured by Sakai Chemical Industry Co., Ltd.);
Zinc oxide/hydrous silica/dimethicone (Ultrafine Zinc Oxide FINEX (registered trademark)-51W-LP3 manufactured by Sakai Chemical Industry Co., Ltd.);
Carboxylic acid-modified silicone (ES-5800 formulation aid manufactured by Dow Toray Co., Ltd.); and
Triethanolamine (Triethanolamine Care manufactured by BASF).

### <Absorbance>

A sample was dropped onto a measurement plate (S plate) (5×5 cm V-groove PMMA plate; SPFMASTER-PA01) in an amount of 2 mg/cm², applied with a finger for 60 seconds, dried for 15 minutes, and then the absorbance (280 to 400 nm) was measured with a spectrophotometer (U-3500 by Hitachi, Ltd.), using an uncoated plate as a control, and an integrated absorbance value was obtained from the measured data. Note that the results of absorbance are shown in FIG. 5 to FIG. 7.

FIG. 5 shows the absorbance of a water-in-oil emulsion composition A, in which a cationic surfactant was used as the dispersant for the aqueous slurry, an ultraviolet protective agent was added, and a hydrophobic powder was dispersed in both the external oil phase and the internal aqueous phase, and the absorbance of a water-in-oil emulsion composition B, in which a UV protection agent was added, and a hydrophobic powder was dispersed only in the external oil phase.

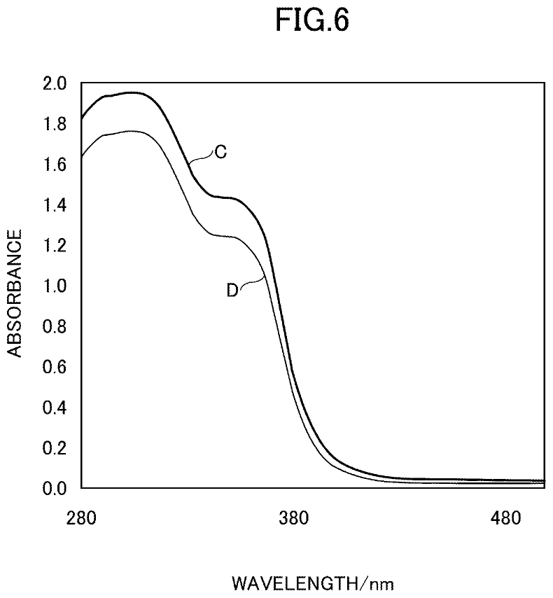

FIG. 6 shows the absorbance of a water-in-oil emulsion composition C, in which a cationic surfactant was used as the dispersant for the aqueous slurry, no ultraviolet protective agent was added, and a hydrophobic powder was dispersed in both the external oil phase and the internal aqueous phase, and the absorbance of a water-in-oil emulsion composition D, in which no ultraviolet protective agent was added, and a hydrophobic powder was dispersed only in the external oil phase.

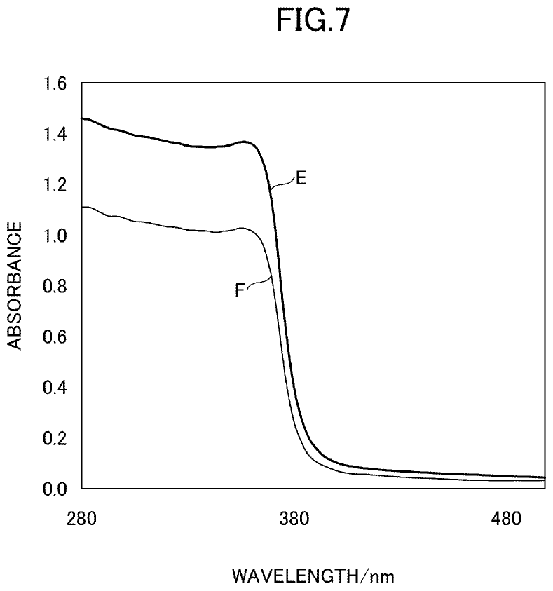

FIG. 7 shows the absorbance of a water-in-oil emulsion composition E, in which a silicone-based surfactant was used as the dispersant for the aqueous slurry, an ultraviolet protective agent was added, and a hydrophobic powder was dispersed in both the external oil phase and the internal aqueous phase, and the absorbance of a water-in-oil emulsion composition F, in which a UV protection agent was added, and a hydrophobic powder was dispersed only in the external oil phase.

Regarding the criteria of evaluation for absorbance, if the absorbance value of an embodiment was higher than that of a comparative example, the absorbance of the embodiment was evaluated as “∘” (good). For example, in the example shown in FIG. 5, A was evaluated as “∘” (good) compared to B; in the example shown in FIG. 6, C was evaluated as “∘” (good) compared to D; and, in the example shown in FIG. 7, E was evaluated as “∘” (good) compared to F.

### <SPF>

A sample was dropped in an amount of 2 mg/cm² onto a measurement plate (S plate) (5×5 cm V-groove PMMA plate, SPFMASTER-PA01), applied with a finger for 60 seconds, dried for 15 minutes, and then the sun protection factor (SPF) or the ultraviolet B wave (UVB) protection index was measured in vitro by using an SPF measurement device (SPF MASTER (registered trademark) manufactured by Shiseido Irika Technology Inc.).

SPF was evaluated according to the following criteria. Note that embodiment 1 was evaluated against comparative examples 1 to 3, example 2 was evaluated against comparative examples 4 to 8, and example 3 was evaluated against comparative examples 9 to 11.
∘: High
Δ: Standard
x : Sample could not be prepared

Embodiments and comparative examples will be described below.

Table 1 shows the formulations and evaluations of embodiments 1 and 2 and comparative examples 1 to 8. Note that Table 1 shows the manufacturers, product names (ingredient names), and so forth, of the ingredients of embodiments 1 and 2 and comparative examples 1 to 8.

### [Table 1]

TABLE 1

| | | | | | Com- | Com- | Com- | Com- | Com- | Com- | Com- | Com- |
|---|---|---|---|---|---|---|---|---|---|---|---|---|
| | | | | | parative | parative | parative | parative | parative | parative | parative | parative |
| | | | Embodi- | Embodi- | Example | Example | Example | Example | Example | Example | Example | Example |
| Composition | | | ment 1 | ment 2 | 1 | 2 | 3 | 4 | 5 | 6 | 7 | 8 |
| For- | Solvent | Water | — | — | 25 | — | — | 24 | — | — | — | — |
| mulation | Thickener | Disteardimonium Hectorite | 0.3 | 0.6 | 0.3 | 0.3 | 0.3 | 0.6 | 0.6 | 0.6 | 0.6 | 0.6 |
| (Part) | Dispersant | PEG-9 | 1.5 | 2 | 1.5 | 1.2 | 1.5 | 2 | 1.7 | 2 | 2 | 2 |
| | | Polydimethylsiloxyethyl | | | | | | | | | | |
| | | Dimethicone/Tocopherol | | | | | | | | | | |
| | | Distearyldimonium Chloride | — | — | 0.3 | 0.3 | 0.3 | 0.3 | 0.3 | 0.3 | 0.3 | 0.3 |
| | | Isostearic Acid | 0.45 | 0.5 | 0.45 | 0.45 | 0.15 | 0.5 | 0.5 | 0.2 | 0.5 | 0.5 |
| | | PEG-10 Dimethicone | — | 2 | — | — | — | 2 | 2 | 2 | 1.7 | 2 |
| | | Bisbutyl Dimethicone | — | 2 | — | — | — | 2 | 2 | 2 | 2 | 1.7 |
| | | Polyglyceryl-3 | | | | | | | | | | |
| | | Diisopropyl Sebacate | 1 | — | 1 | — | 1 | — | — | — | — | — |
| | | Dimethicone* | 17.94 | 31.6 | 17.94 | 17.94 | 17.94 | 31.6 | 31.6 | 31.6 | 31.6 | 31.6 |
| | | Dimethicone** | 3.5 | — | 3.5 | 3.5 | 3.5 | — | — | — | — | — |
| | Oil | Stearic Acid | 0.01 | — | 0.01 | 0.01 | 0.01 | — | — | — | — | — |
| | Com- | Isopropyl Myristate | 5 | 10 | 5 | 5 | 5 | 10 | 10 | 10 | 10 | 10 |
| | ponent | Isododecane | 3 | — | 3 | 3 | 3 | — | — | — | — | — |
| | | PPG-17/BHT | 1 | — | 1 | 1 | 1 | — | — | — | — | — |
| | | Trimethylsiloxysilicate/ | 1 | — | 1 | 1 | 1 | — | — | — | — | — |
| | | Dimethicone | | | | | | | | | | |
| | | Dextrin Palmitate | 1 | — | 1 | 1 | 1 | — | — | — | — | — |
| | Ultra- | Zinc Oxide/ | 9 | 14 | 13 | 9 | 9 | 20 | 14 | 14 | 14 | 14 |
| | violet | Triethoxycaprylylsilane | | | | | | | | | | |
| | Protection | Octocrylene | 3 | — | 3 | 3 | 3 | — | — | — | — | — |
| | Agent | Bisethylhexyloxyphenol | 0.5 | — | 0.5 | 0.5 | 0.5 | — | — | — | — | — |
| | | Methoxyphenyltriazine | | | | | | | | | | |
| | | Diethylaminohydroxybenzoyl | 1.5 | — | 1.5 | 1.5 | 1.5 | — | — | — | — | — |
| | | Hexyl Benzoate | | | | | | | | | | |
| | | Ethylhexyl | 7.5 | — | 7.5 | 7.5 | 7.5 | — | — | — | — | — |
| | | Methoxycinnamate/BHT | | | | | | | | | | |
| | Powder | (Vinyl Dimethicone/ | 1 | — | 1 | 1 | 1 | — | — | — | — | — |
| | | Methicone Silsesquioxane) | | | | | | | | | | |
| | | Crosspolymer | | | | | | | | | | |
| | | Talc/Dimethicone | 7 | 7 | 7 | 7 | 7 | 7 | 7 | 7 | 7 | 7 |
| | | Methyl Methacrylate | 4.5 | — | 4.5 | 4.5 | 4.5 | — | — | — | — | — |
| | | Crosspolymer | | | | | | | | | | |
| | | Silica | 1 | — | 1 | 1 | 1 | — | — | — | — | — |
| | Aqueous | Water | 25 | 24 | — | 25 | 25 | — | 24 | 24 | 24 | 24 |
| | Slurry | Zinc Oxide/ | 4 | 6 | — | 4 | 4 | — | 6 | 6 | 6 | 6 |
| | | Triethoxycaprylylsilane | | | | | | | | | | |
| | | Distearyldimonium Chloride | 0.3 | 0.3 | — | — | — | — | — | — | — | — |
| | | PEG-9 | — | — | — | 0.3 | — | — | 0.3 | — | — | — |
| | | Polydimethylsiloxyethyl | | | | | | | | | | |
| | | Dimethicone | | | | | | | | | | |
| | | Isostearic Acid | — | — | — | — | 0.3 | — | — | 0.3 | — | — |
| | | PEG-10 Dimethicone | — | — | — | — | — | — | — | — | 0.3 | — |
| | | Bisbutyl Dimethicone | — | — | — | — | — | — | — | — | — | 0.3 |
| | | Polyglyceryl-3 | | | | | | | | | | |

| | | | | | | | | | | | |
|---|---|---|---|---|---|---|---|---|---|---|---|
| | Total | 100 | 100 | 100 | 100 | 100 | 100 | 100 | 100 | 100 | 100 |
| Evaluation | SPF | ○ | ○ | Δ | x | x | Δ | x | x | x | x |

Table 2 shows the formulations and evaluations of embodiment 3 and comparative examples 9 to 11. Note that Table 2 shows the manufacturers, product names (ingredient names), and so forth, of the ingredients of example 3 and comparative examples 9 to 11.

### [Table 2]

TABLE 2

| | | | Embodi- | Com- | Com- | Com- |
|---|---|---|---|---|---|---|
| | | | ment | parative | parative | parative |
| Composition | | | 3 | Example 9 | Example 10 | Example 11 |
| For- | Solvent | Water | 1.2 | 15.5 | 2.6 | 2.6 |
| mulation | | Glycerin | 5 | 5 | 5 | 5 |
| (Part) | | BG | 5 | 5 | 5 | 5 |
| | Thickener | Disteardimonium Hectorite | 0.3 | 0.3 | 0.3 | 0.3 |
| | Dispersant | PEG-9 | 2 | 2 | 1 | 2 |
| | | Polydimethylsiloxyethyl | | | | |
| | | Dimethicone | | | | |
| | | Bisbutyl Dimethicone | 1.5 | 1.5 | 1.5 | 0.5 |
| | | Polyglyceryl-3 | | | | |
| | Oil | Cyclopentasiloxane | 33 | 34.7 | 33 | 33 |
| | Com- | Dimethicone** | 5 | 5 | 5 | 5 |
| | ponent | Dimethicone*** | 2 | 2 | 2 | 2 |
| | | Diphenylsiloxyphenyl | 4 | 4 | 4 | 4 |
| | | Trimethicone | | | | |
| | Ultraviolet | Titanium Oxide/Aluminum | 10 | 10 | 10 | 10 |
| | Protection | Hydroxide/Hydrogen | | | | |
| | Agent | Dimethicone | | | | |
| | | Zinc Oxide/Hydrous Silica/ | 6 | 10 | 6 | 6 |
| | | Dimethicone | | | | |
| | Powder | Methyl Methacrylate | 5 | 5 | 5 | 5 |
| | | Crosspolymer | | | | |
| | Aqueous | Water | 11.2 | — | 11.2 | 11.2 |
| | Slurry | Zinc Oxide/ | 6 | — | 6 | 6 |
| | | Triethoxycaprylylsilane | | | | |
| | | BG | 1.4 | — | 1.4 | 1.4 |
| | | Carboxylic Acid-Modified | 1 | — | — | — |
| | | Silicone | | | | |
| | | Triethanolamine | 0.4 | — | — | — |
| | | PEG-9 | — | — | 1 | — |
| | | Polydimethylsiloxyethyl | | | | |
| | | Dimethicone | | | | |
| | | Bisbutyl Dimethicone | — | — | — | 1 |
| | | Polyglyceryl-3 | | | | |

| | | | | | |
|---|---|---|---|---|---|
| | Total | 100 | 100 | 100 | 100 |
| Evaluation | SPF | ○ | Δ | x | x |

According to FIG. 5, FIG. 6, and Table 1, water-in-oil emulsion compositions, in which a metal oxide treated with trimethylethoxysilane as a hydrophobic powder was dispersed in both the external oil phase and the internal aqueous phase, and in which a cationic surfactant was contained as the dispersant for the aqueous slurry, had both improved absorbance and SPF (examples 1 and 2).

Furthermore, according to FIG. 7 and Table 2, a water-in-oil emulsion composition containing a silicone-based surfactant as the dispersant for the aqueous slurry had improved absorbance and good SPF (embodiment 3).

On the other hand, no improvement in absorbance and SPF was confirmed with water-in-oil emulsion compositions in which a hydrophobic powder was dispersed in the external oil phase alone (comparative examples 1 to 11).

From these results, it was found out that a water-in-oil emulsion composition: which had an external oil phase in which a first hydrophobic powder was dispersed and an internal aqueous phase in which a second hydrophobic powder was dispersed; in which at least the second hydrophobic powder among the first hydrophobic powder and the second hydrophobic powder was a metal oxide treated with trimethylethoxysilane; which contained a dispersant that dispersed the second hydrophobic powder in the internal aqueous phase; and in which the dispersant was at least one of a silicone-based surfactant and a cationic surfactant, had a potential for improving various functions of cosmetics.

Although embodiments of the present invention have been described above, the present invention is not limited to specific embodiments, and various modifications and changes can be made within the scope of the invention described in the claims.

This application claims priority based on Japanese Patent Application No. 2021-55610, filed on Mar. 29, 2021, the entire contents of which are incorporated herein by reference.

### REFERENCE SIGNS LIST

100 water-in-oil emulsion composition
10 external oil phase (continuous phase)
11 first hydrophobic powder
20 internal aqueous phase (dispersed phase)
21 second hydrophobic powder
S skin
F finger

1. A water-in-oil emulsion composition comprising:
an external oil phase, in which a first hydrophobic powder is dispersed; and
an internal aqueous phase, in which a second hydrophobic powder is dispersed,
wherein, among the first hydrophobic powder and the second hydrophobic powder, at least the second hydrophobic powder is a metal oxide treated with trimethylethoxysilane,
wherein the water-in-oil emulsion composition further comprises a dispersant that disperses the second hydrophobic powder in the internal aqueous phase, and
wherein the dispersant is at least one of a silicone-based surfactant and a cationic surfactant.

2. The water-in-oil emulsion composition according to claim 1,
wherein the internal aqueous phase contains an aqueous slurry, and
wherein the aqueous slurry contains the second hydrophobic powder, an aqueous medium, and the dispersant that disperses the second hydrophobic powder in the aqueous medium.

3. The water-in-oil emulsion composition according to claim 2, wherein the aqueous slurry is obtained by dispersing the second hydrophobic powder in a mixed solvent of the dispersant and the aqueous medium.

4. The water-in-oil emulsion composition according to claim 2, wherein the aqueous slurry is obtained by dispersing a mixture of the second hydrophobic powder and the dispersant in the aqueous medium.

5. The water-in-oil emulsion composition according to claim 1, wherein the silicone-based surfactant is a mixture of a carboxylic acid-modified silicone and an alkanolamine.

6. The water-in-oil emulsion composition according to claim 1, wherein the cationic surfactant is a dialkyldimethylammonium chloride represented by the following general formula: 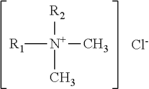
where R1 and R2 are hydrocarbon groups having 10 to 22 carbon atoms.

7. The water-in-oil emulsion composition according to claim 1, further comprising an emulsifier.

8. The water-in-oil emulsion composition according to claim 1, further comprising a thickener.

9. The water-in-oil emulsion composition according to claim 1, further comprising a moisturizer.

10. A cosmetic comprising the water-in-oil emulsion composition according to claim 1.

11. A method for preparing a water-in-oil emulsion composition in which an aqueous phase is dispersed in an oil phase, the method comprising:
a first step of dispersing a first hydrophobic powder in the oil phase; and
a second step of dispersing a second hydrophobic powder in the aqueous phase, wherein the second step further includes:
obtaining a mixture by heating and dissolving the second hydrophobic powder, a dispersant, and an alcohol at 80° C. to 90° C.;
dispersing the mixture in water to produce an aqueous slurry; and
dispersing the aqueous slurry in the aqueous phase.
